(19) United States
(12) Patent Application Publication
(10) Pub. No.: US 2014/0058435 A1
(43) Pub. Date: Feb. 27, 2014

(54) IMPLANT DELIVERY AND RELEASE SYSTEM

(71) Applicants: Donald K. Jones, Dripping Springs, TX (US); Vladimir Mitelberg, Austin, TX (US)

(72) Inventors: Donald K. Jones, Dripping Springs, TX (US); Vladimir Mitelberg, Austin, TX (US)

(21) Appl. No.: 13/971,828

(22) Filed: Aug. 20, 2013

Related U.S. Application Data
(60) Provisional application No. 61/691,478, filed on Aug. 21, 2012.

(51) Int. Cl. A61B 17/12

(52) U.S. Cl. CPC A61B 17/1214
USPC 606/200

(57) ABSTRACT

A medical implant deployment system for placing an implant at a preselected site within a vessel, duct or body lumen of a mammal comprising a reloadable deployment system includes a mechanical coupling assembly at the distal end of a delivery member, having an extended configuration in which the coupling assembly is adapted to receive or release the implant proximal end and a retracted configuration where the coupling assembly distal end is interlockingly engaged with the implant proximal end. Once the implant is properly positioned the coupling assembly is actuated, thereby releasing the implant at a desired position within the body.

### BACKGROUND OF THE INVENTION

For many years flexible catheters have been used to place various devices within the vessels of the human body. Such devices include dilatation balloons, radio-opaque fluids, liquid medications and various types of occlusion devices such as balloons and embolic coils. Examples of such catheter devices are disclosed in U.S. Pat. No. 5,108,407, entitled “Method And Apparatus For Placement Of An Embolic Coil”; U.S. Pat. No. 5,122,136, entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils to preselected positions within vessels of the human body in order to treat aneurysms, or alternatively, to occlude the blood vessel at the particular location.

Coils which are placed in vessels may take the form of helically wound coils, or alternatively, may be random wound coils, coils wound within other coils or many other such configurations. Examples of various coil configurations are disclosed in U.S. Pat. No. 5,334,210, entitled, “Vascular Occlusion Assembly; U.S. Pat. No. 5,382,259, entitled, “Vasoocclusion Coil With Attached Tubular Woven Or Braided Fibrous Coverings.” Embolic coils are generally formed of radiopaque metallic materials, such as platinum, gold, tungsten, or alloys of these metals. Often times, several coils are placed at a given location in order to occlude the flow of blood through the vessel by promoting thrombus formation at the particular location.

In the past, embolic coils have been placed within the distal end of the catheter. When the distal end of the catheter is properly positioned the coil may then be pushed out of the end of the catheter with, for example, a guidewire to release the coil at the desired location. This procedure of placement of the embolic coil is conducted under fluoroscopic visualization such that the movement of the coil through the vasculature of the body may be monitored and the coil may be placed at the desired location. With these placements systems there is very little control over the exact placement of the coil since the coil may be ejected to a position some distance beyond the end of the catheter.

Numerous procedures have been developed to enable more accurate positioning of coils within a vessel. Still another such procedure involves the use of a glue, or solder, for attaching the embolic coil to a guidewire which, is in turn, placed within a flexible catheter for positioning the coil within the vessel at a preselected position. Once the coil is at the desired position, the coil is restrained by the catheter and the guidewire is pulled from the proximal end of the catheter to thereby cause the coil to become detached from the guidewire and released from the catheter system. Such a coil positioning system is disclosed in U.S. Pat. No. 5,263,964, entitled, “Coaxial Traction Detachment Apparatus And Method.”

Another coil positioning system utilizes a catheter having a socket at the distal end of the catheter for retaining a ball which is bonded to the proximal end of the coil. The ball, which is larger in diameter than the outside diameter of the coil, is placed in a socket within the lumen at the distal end of the catheter and the catheter is then moved into a vessel in order to place the coil at a desired position. Once the position is reached, a pusher wire with a piston at the end thereof is pushed distally from the proximal end of the catheter to thereby push the ball out of the socket in order to release the coil at the desired position. Such a system is disclosed in U.S. Pat. No. 5,350,397, entitled, “Axially Detachable Embolic Coil Assembly.” One problem with this type of coil placement system which utilizes a pusher wire which extends through the entire length of the catheter and which is sufficiently stiff to push an attachment ball out of engagement with the socket at the distal end of the catheter is that the pusher wire inherently causes the catheter to be very stiff with the result that it is very difficult to guide the catheter through the vasculature of the body.

Yet another coil deployment system is disclosed in U.S. Pat. No. 5,261,916, entitled, “Detachable Pusher-Vasooclusive Coil Assembly with Interlocking Ball and Keyway Coupling.” This system includes a pusher member with a tubular portion at its distal end that has a keyway for receiving the enlarged bead of an embolic coil through the outer wall and into the lumen of the tubular portion. The enlarged bead of the coil is positioned within the keyway and a resilient wire coupling the bead to the coil extends axially over the outer diameter of the distal end of the tubular portion to the remaining portion of the coil. The enlarged bead is retained in the keyway, forming an interlocking arrangement, by positioning the assembly within the lumen of an outer sleeve. Once the keyway is pushed from the confines of the sleeve the bead can disengage from the keyway. With this system the inner diameter has to be sufficiently large to accommodate the stack up of the wire coupled to the bead and the diameter of the tubular portion. Also when placing coils in an aneurysm “packed” with coils, there may not be enough room for the enlarged bead to disengage from the keyway.

Another coil release system is disclosed in U.S. Pat. No. 5,895,391 to Farnholtz, entitled, “Ball Lock Joint and Introducer for Vaso-occlusive Member”. This system incorporates a tubular member having a portion of the wall cut away to receive at least a portion of an enlarged bead coupled to the proximal end of the embolic coil. A wire is placed within the lumen of the tubular member and cooperates to form an interference fit between the wire, bead and cut-away wall portion. To release the coil, the wire is pulled from the proximal end of the system to remove the interference fit with the bead and cut-away wall portion.

Still another coil deployment system utilizes a pair of jaws placed on the distal end of a pusher wire to position and release a coil. One such system is described in U.S. Pat. Nos. 5,601,600 and 5,746,769 to Ton et al., entitled, “Endoluminal Coil Delivery System Having A Mechanical Release Mechanism.” Ton discloses an elongate pusher wire having jaws at the distal end. The jaws include tip projections which are perpendicular to the longitudinal axis of the pusher wire and when positioned with the lumen of a collar fixed to the proximal end of a coil, interlockingly engage with matching detents placed in the wall of the collar. A tubular body is used to slide over the pusher wire to collapse the jaws and release the collar. The disclosed interlocking engagement between the jaws and collar prevents forward and backwards axial movement of the jaws relative to the collar and allows any torquing force applied to the jaws to be translated to the collar and affixed coil. Transmission of torque from a coil delivery system to a coil during the treatment of aneurysm may be detrimental to precise placement of the coil. The coils may coils store the torque energy and upon release from the delivery system, release the stored energy causing the coils to move unpredictably. Ton also states that jaws may be fixed to the coil, but does not provide or disclose any information as to how this may be accomplished.

Another method for placing an embolic coil is that of utilizing a heat releasable adhesive bond for retaining the coil at the distal end of the catheter. One such system uses laser energy which is transmitted through a fiber optic cable in order to apply heat to the adhesive bond in order to release the coil from the end of the catheter. Such a method is disclosed in U.S. Pat. No. 5,108,407, entitled, “Method And Apparatus For Placement Of An Embolic Coil.” Such a system also suffers from the problem of having a separate, relatively stiff element which extends throughout the length of the catheter with resulting stiffness of the catheter.

Another method for placing an embolic coil is that of utilizing a heat responsive coupling member which bonds the coil to the distal end of a delivery system. One such system uses electrical energy which is transmitted through electrical conductors to create heat which is applied to the coupling member to thereby soften and yield the coupling member in order to release the coil from the end of the delivery system. Such a method is disclosed in U.S. Pat. No. 7,179,276, entitled, “Heated Vascular Occlusion Coil Deployment System.” Such a system suffers from the problem of having to pull an engagement member once the coupling is softened in order to release the coil.

### BRIEF SUMMARY OF THE INVENTION

The present invention is directed toward a medical implant deployment system for use in placing a medical implant at a preselected site within the body of a mammal which includes an elongate delivery system having a coupling assembly at its distal end that releasably engages the proximal end of a medical implant. Typical medical implants include devices such as stents, filters, vascular plugs, aneurysm embolization devices, embolic coils and flow diverters configured for delivery through a catheter. The delivery system includes an elongate tubular delivery member having proximal and distal ends. A coupling assembly is positioned at the distal end of the delivery member and includes a tubular tip element fixedly coupled to the distal end of the delivery member, a tubular retaining member having a retaining portion positioned at the distal end of the retaining member and an elongate flexible actuator member having proximal and distal ends positioned within the lumen of the delivery member. The actuator member distal end is fixedly coupled to the retaining member proximal end. The coupling assembly has a refracted configuration in which the retaining portion of the retaining member and the tip element cooperatively engage the proximal end of the medical implant to restrict distal movement of the implant relative to the coupling assembly. The coupling assembly also has an extended configuration wherein the retaining portion of the retaining member extends distal to the tip element to accept or release the proximal end of the medical implant.

The delivery system along with the distally located and releasably coupled medical implant are slidably positioned within the lumen of a catheter whose distal end is positioned adjacent a target implantation site. The delivery system is advanced such that the implant proximal end and coupling assembly distal end exit the lumen of the catheter. Once the implant is in the desired location, the coupling assembly is moved from its retracted configuration to its extended configuration in which the actuator member is advanced distally, relative to the delivery member, causing the retaining portion to move distally from the tip element distal end, thus removing the cooperative engagement of the retaining portion and tip element that previously restricted distal movement of the implant. While in the extended configuration the coupling assembly is moved proximally to insure the retaining portion is completely disengaged from the proximal end of the medical implant, thereby releasing the implant at the target site.

The tubular delivery member is formed utilizing construction techniques well known in the formation of catheters or microcatheters. These construction techniques include for example braiding, coiling, extruding, laser cutting, joining, crimping, laminating, fusing and welding of components (such as markers bands, distal coil sections, tips and proximal sections) or portions of components to provide a tubular member having sufficient pushability, visibility and flexibility to traverse the luminal tortuosity when accessing an intended implantation site.

In accordance with an aspect of the present invention, there is provided a medical implant that takes the form of an embolization device such as an embolic or vaso-occlusive coil for selective placement within a vessel, aneurysm, duct or other body lumen. Embolic coils are typically formed through the helical winding of a filament or wire to form an elongate primary coil. The wire or filament is typically a biocompatible material suitable for implantation and includes metals such as platinum, platinum alloys, stainless steel, nitinol and gold. Other biocompatible materials such as plastics groups including nylons, polyesters, polyolefins and fluoro-polymers may be processed produce suitable filaments for forming coils. The wire usually has a circular cross-section, however, non-circular cross-sections, such as “D” shapes, are used in commercially available coils. The diameter of the wire may range from 0.0001″ to about 0.010″ and is largely dependent upon the particular clinical application for the coil. The diameter of the primary coil is generally dependent upon the wire diameter and the diameter of the mandrel used for winding. The primary coil diameter typically ranges from 0.002″ to about 0.060″ and is also dependent upon on the clinical application. The wound primary coil is typically removed from the mandrel leaving the coil with a lumen. In addition to the aforementioned method of winding a coil, there are other “mandrel-less” forming processes that are suitable for making primary coils that plastically deform the wire into coil. The formed primary coils may be further processed to have a secondary shape such as a helix, sphere, “flower”, spiral or other complex curved structure suited for implantation in a particular anatomical location. The secondary shape is imparted to the coil through thermal or mechanical means. Thermal means include forming the primary coil into a desired shape using a die or forming tool and then heat treating the coil to retain the secondary shape. Mechanical means include plastically deforming the primary coil into the desired shape or the use of a shaped resilient core wire inserted into the lumen of the primary coil to impart a shape to the coil. The length of the elongate primary coil range from 0.1 cm to about 150 cm with a preferred range of about 0.5 cm to about 70 cm. The distal end of the coil is typically rounded or beaded to make the coil end more atraumatic. Other variations of embolic coils suitable for use include stretch resistant coils, coils that incorporate a stretch resistant member(s) (within the coil lumen or exterior to the coil) that limits undesirable elongation of the primary coil during device manipulation and coated or modified coils that enhance occlusion through coils surface modifications, addition of therapeutics or volume filling materials (foams, hydrogels, etc.).

In accordance with another aspect of the present invention, the proximal end of the implant may include an engagement member. The engagement member is fixedly coupled to the implant and positioned at the proximal end of the implant. For example, an implant that takes the form of an elongate embolic coil may have an engagement member fixedly attached to the proximal end of the coil or positioned at the proximal end of the coil and fixedly attached to another location on the coil. The engagement member typically takes the form of an enlarged bead; however, any other forms compatible with the retaining portion of the retaining member and the tip element may be suitable.

In accordance with yet another aspect of the present invention, the retaining portion of the retaining member includes an aperture. The engagement member of the medical implant may be partially received within the aperture of the retaining portion when the coupling assembly of the delivery system is in the retracted configuration.

In accordance with another aspect of the present invention there is provided a delivery system that includes a proximal spring member positioned proximal to the distal end of the delivery member. The proximal spring member has proximal and distal ends and is coaxially positioned about the actuator member such that the actuator member extends through the lumen of the proximal spring member. The proximal spring member distal end is coupled to the delivery member and the proximal end of the spring member coupled to the actuator member. The proximal spring member is preferably positioned at the proximal end of the delivery member; however the proximal spring member may be positioned distal to the proximal end of the delivery member. The proximal spring member is preferably biased to place the coupling assembly of the delivery system in a retracted configuration where the retaining portion of the retaining member is positioned within the lumen of the tip element to cooperatively engage the proximal end of a medical implant.

In accordance with an additional aspect of the present invention there is provided a delivery system that includes a distal spring member positioned within the lumen of the distal end of the delivery member. The distal spring member has proximal and distal ends and is coaxially positioned about the actuator member distal end such that the actuator member extends through the lumen of the distal spring member. The distal spring member distal end is fixedly coupled to the retaining member. The proximal end of the distal spring member is restricted from moving proximally relative to the delivery member and may optionally be fixedly coupled to the delivery member. The distal spring member is preferably biased to place the coupling assembly of the delivery system in an extended configuration where the retaining portion of the retaining member extends distal to the tip element and is in a position to receive or release the proximal end of a medical implant.

In accordance with another aspect of the present invention there is provided a coil introducer assembly that facilitates both the coupling of an embolic coil proximal end with the distal end of the delivery system and the transfer of the delivery system with a coupled coil to the lumen of a catheter. The coil introducer assembly includes an elongate tubular introducer having proximal and distal ends, a securing region positioned between the proximal and distal ends and a lumen extending therethrough, an elongate embolic coil having proximal and distal ends positioned within the lumen of the introducer and a securing member coupled to the introducer securing region. The proximal end of the elongate coil is positioned within the securing region of the introducer such that the proximal end of the coil is directed towards the proximal end of the introducer. The securing member has a first configuration in which the wall of the introducer in the securing region is compressed such that the coil proximal end positioned within the securing region encounters substantial resistance to axial movement. The securing member also has a second configuration in which the wall of the introducer in the securing region is uncompressed such that the coil proximal end positioned within the securing region encounters minimal resistance to axial movement. The securing member preferably takes the form of a perforated heat shrink tubing having a tab member in which the heat shrink tubing has been positioned on the introducer and shrunk to retain the proximal end of the coil within the securing region of the introducer in the first configuration. To move the securing member to the second configuration, a pull force is applied to the tab member of the heat shrink tubing to tear the heat shrink wall along the perforations and remove the compressive force applied to the securing region, thereby allowing the proximal end of the coil to move axially encountering only with minimal resistance. The securing member may take other suitable forms including hemostasis valves, elastic bands, and other releasable clamping structures.

In accordance with yet another aspect of the present invention there is provided a method of delivering an implant at a target site that includes: providing a delivery system having a coupling assembly; providing a medical implant having a proximal end adapted to engage the distal end of the delivery system; verifying that the coupling assembly of the delivery system is placed in an extended configuration; inserting the proximal end of the medical implant into the distal end of the coupling assembly; operating the delivery system to place the coupling assembly in a retracted configuration; verifying that the delivery system appropriately engages the implant; positioning the medical implant and delivery system within the lumen of a catheter having a distal end adjacent to a target implant site; advancing the delivery system through the catheter such that the implant exits the catheter lumen at its distal end; positioning the implant in a desired location; operating the delivery system to place the coupling assembly in an extended configuration; removing the coupling assembly distal end from proximal end of the medical implant to thereby release the implant; and removing the delivery system from the catheter lumen.

In accordance with another aspect of the present invention there is provided a method for delivering additional implants using the same delivery system that further includes: providing an additional medical implant having a proximal end adapted to engage the distal end of the delivery system; verifying that the coupling assembly of the delivery system is placed in an extended configuration; inserting the proximal end of the additional medical implant into the distal end of the coupling assembly; operating the delivery system to place the coupling assembly in a retracted configuration; verifying that the delivery system appropriately engages the additional implant; positioning the additional medical implant and delivery system within the lumen of a catheter having a distal end adjacent to a target implant site; advancing the delivery system through the catheter such that the additional implant exits the catheter lumen at its distal end; positioning the additional implant in a desired location; operating the delivery system to place the coupling assembly in an extended configuration; removing the coupling assembly distal end from the proximal end of the additional medical implant; and removing the delivery system from the catheter lumen.

In accordance with still yet another aspect of the present invention there is provided a method of delivering an embolic coil to a target site that includes: providing a delivery system having a coupling assembly; providing a coil introducer assembly having a coil with a proximal end adapted to engage the distal end of the delivery system; inserting the distal end of the delivery system into the lumen of the coil introducer assembly proximal end; verifying that the coupling assembly of the delivery system is placed in an extended configuration; advancing the distal end of the delivery system distally such that the coupling assembly contacts the proximal end of the embolic coil; operating the delivery system to place the coupling assembly in a retracted configuration; verifying that the delivery system appropriately engages the embolic coil; operating the securing member of the coil introducer assembly to allow the coil to move axially within the introducer with minimal resistance; positioning the distal end of the coil introducer adjacent the lumen of the catheter at the catheter proximal end; advancing the delivery system and engaged coil distally through the introducer lumen to thereby exit the lumen at the distal end of the introducer and enter the lumen at the proximal end of the catheter; positioning the delivery system and engaged coil within the lumen of a catheter having a distal end adjacent to a target site; advancing the delivery system through the catheter such that the coil exits the catheter lumen at its distal end; positioning the coil in a desired location; operating the delivery system to place the coupling assembly in an extended configuration; removing the coupling assembly distal end from proximal end of the coil to thereby release the coil; and removing the delivery system from the catheter lumen.

In accordance with still yet another additional aspect of the present invention there is provided a method of delivering additional embolic coils using the same delivery system that further includes: providing an additional coil introducer assembly having a coil with a proximal end adapted to engage the distal end of the delivery system; inserting the distal end of the delivery system into the lumen of the additional coil introducer assembly proximal end; verifying that the coupling assembly of the delivery system is placed in an extended configuration; advancing the distal end of the delivery system distally such that the coupling assembly contacts the proximal end of the additional embolic coil; operating the delivery system to place the coupling assembly in a retracted configuration; verifying that the delivery system appropriately engages the additional embolic coil; operating the securing member of the additional coil introducer assembly to allow the additional coil to move axially within the additional introducer with minimal resistance; positioning the distal end of the additional coil introducer adjacent the lumen of the catheter at the catheter proximal end; advancing the delivery system and engaged additional coil distally through the introducer lumen to thereby exit the lumen at the distal end of the introducer and enter the lumen at the proximal end of the catheter; positioning the delivery system and engaged additional coil within the lumen of a catheter having a distal end adjacent to a target site; advancing the delivery system through the catheter such that the additional coil exits the catheter lumen at its distal end; positioning the additional coil in a desired location; operating the delivery system to place the coupling assembly in an extended configuration; removing the coupling assembly distal end from proximal end of the additional coil to thereby release the additional coil; and removing the delivery system from the catheter lumen.

These aspects of the invention and the advantages thereof will be more clearly understood from the following description and drawings of embodiments of the present invention:

### BRIEF DESCRIPTION OF THE DRAWINGS

FIG. 1 is a partially sectioned view of a medical implant deployment system according to an embodiment of the present invention.

FIG. 2 is an enlarged partially sectioned view showing a distal portion of the medical implant deployment system of FIG. 1.

FIG. 3A is a partial perspective view of the delivery system distal end in an extended configuration.

FIG. 3B is a partial perspective view of the delivery system distal end in a retracted configuration.

FIG. 4 is a partially sectioned view of the delivery system distal end in a retracted configuration engaged with an embolic coil.

FIG. 5 is a partially sectioned view of the delivery system distal end in a partially extended configuration engaged with the embolic coil.

FIG. 6 is a partially sectioned view of the delivery system distal end in an extended configuration releasing the embolic coil.

FIG. 7 is a partially sectioned view of the delivery system distal end in an retracted configuration after release of the embolic coil.

FIG. 8 is a partially sectioned view of the delivery system distal end in a retracted configuration engaged with an embolic coil according to another embodiment of the present invention.

FIG. 9 is a partially sectioned view of the delivery system distal end in a retracted configuration engaged with an embolic coil according to yet another embodiment of the present invention.

FIG. 10 is a partially sectioned view of a medical implant deployment system according to another embodiment of the present invention.

FIG. 11 is an enlarged partially sectioned view showing a distal portion of the medical implant deployment system of FIG. 10.

FIG. 12 is a partially sectioned view of the delivery system distal end in a retracted configuration engaged with a partially sectioned coil.

FIG. 13 is a partially sectioned view of the delivery system distal end in a partially extended configuration engaged with the embolic coil.

FIG. 14 is a partially sectioned view of the delivery system distal end in an extended configuration releasing the embolic coil.

FIG. 15 is a partially sectioned view of the delivery system distal end in a retracted configuration after releasing the embolic coil.

FIG. 16 is a partially sectioned view of the coil delivery system and coil introducer assembly according to another embodiment of the invention.

FIG. 17 is an enlarged partially sectioned view of the coil delivery system distal end and proximal end of FIG. 16.

FIG. 18A is a partial perspective view of the coil delivery system distal end in an extended configuration.

FIG. 18B is a partial perspective view of the coil delivery system distal end in a retracted configuration.

FIG. 19 is an enlarged view of the coil introducer assembly of FIG. 16.

FIG. 20 is a partially sectioned view of the coil delivery system distal end in an extended configuration adjacent an embolic coil positioned within the coil introducer.

FIG. 21 is a partially sectioned view of the coil delivery system distal end in a retracted configuration engaged with the embolic coil positioned within the coil introducer.

FIG. 22 is a partially sectioned view of the coil delivery system distal end engaged with the embolic coil positioned within the coil introducer after removal of the securing member.

FIG. 23 is a partially sectioned view of the coil delivery system and engaged embolic coil positioned within the coil introducer being inserted into a catheter.

FIG. 24 is a partially sectioned view of the coil delivery system distal end and engaged embolic coil exiting the distal end of the catheter.

FIG. 25 is a partially sectioned view of the coil delivery system distal end in an extended configuration releasing the embolic coil.

### DETAILED DESCRIPTION OF THE INVENTION

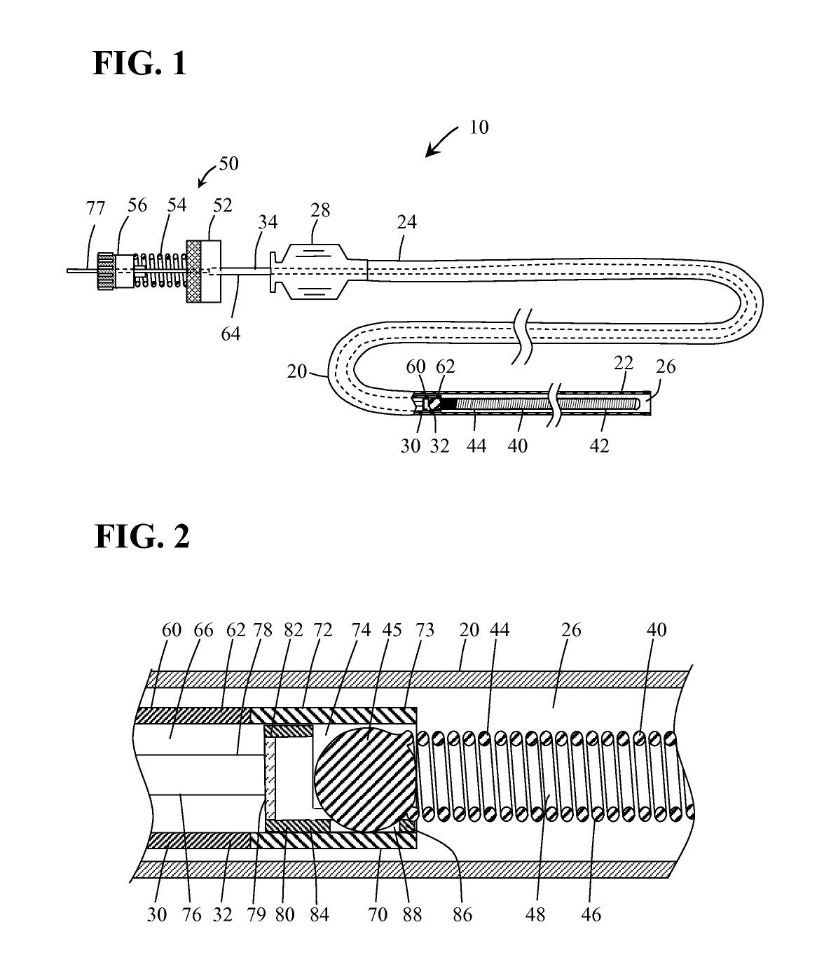

Generally, a medical implant deployment system of the present invention may be used to position an implant at a preselected site within the body of a mammal. The medical implant deployment system may be used to place various implants such as stents, filters, vascular plugs, aneurysm embolization devices, flow diverters and embolization coils. FIG. 1 generally illustrates a medical implant deployment system 10 of the present invention which includes delivery catheter 20 having a distal end 22, a proximal end 24, a lumen 26 extending therethrough and a catheter hub 28 affixed to proximal end 24, a reloadable delivery system 30 having a distal end 32 and a proximal end 34 and an embolic coil 40 having a distal end 42 and a proximal end 44 that is releasably coupled to the distal end 32 of delivery system 30. Embolic coil 40 is a medical implant of a general type suitable for use in occluding a vessel, lumen, duct, opening or aneurysm.

Embolic coil 40 is generally formed from a primary coil of a helically wound wire 46, made from a material which is biocompatible and preferably radio-opaque. Suitable biocompatible materials include metals such as platinum, platinum alloys, stainless steel, nitinol, tantalum and gold and plastics such as nylons, polyesters, polyolefins and fluoropolymers. The wire usually has a circular cross-section, however, non-circular cross-sections, such as “D” shapes, are used in commercially available coils. The diameter of the wire may range from about 0.0001″ to about 0.010″ and is largely dependent upon the particular clinical application for the coil. The diameter of the primary coil is generally dependent upon the wire diameter and the diameter of the mandrel used for winding. The primary coil diameter typically ranges from about 0.002″ to about 0.060″ and is also dependent upon on the clinical application. The wound primary coil is typically removed from the mandrel leaving the coil with a lumen 48. In addition to the aforementioned method of winding a coil, there are other “mandrel-less” forming processes that are suitable for making primary coils that plastically deform the wire into coil. The formed primary coils may be further processed to have a secondary shape such as a helix, sphere, “flower”, spiral or other complex curved structure suited for implantation in a particular anatomical location. The secondary shape is imparted to the coil through thermal or mechanical means. Thermal means include forming the primary coil into a desired shape using a die or forming tool and then heat treating the coil to retain the secondary shape. Mechanical means include plastically deforming the primary coil into the desired shape or the use of a shaped resilient core wire inserted into the lumen of the primary coil to impart a shape to the coil. The length of the elongate primary coil may range from about 0.1 cm to about 150 cm with a preferred range of about 0.5 cm to about 70 cm. The distal end of the coil is typically rounded or beaded to make the coil end more atraumatic. Other variations of embolic coils suitable for use include stretch resistant coils, coils that incorporate a stretch resistant member(s) (within the coil lumen or exterior to the coil) that limits undesirable elongation of the primary coil during device manipulation and coated or modified coils that enhance occlusion through coil surface modifications, addition of therapeutics or volume filling materials (foams, hydrogels, etc.).

As depicted in FIG. 1, deployment system 10 may further include an actuator assembly 50 that is positioned proximal to proximal end 24 of catheter 20. Actuator assembly 50 includes a first coupler member 52, a spacing member 54 and a second coupler member 56. The first and second coupler members 52 and 56 typically take the form of commercially available rotating hemostatic valve (RHV) like assemblies. A typical RHV-like assembly includes a housing body, a threaded cap and a compressible insert. The housing body and threaded cap are typically formed of a rigid plastic such as polystyrene, ABS, nylon or polycarbonate while the insert is formed of an elastomeric material such as silicone or rubber. The assembled housing body, cap and insert all have a contiguous aligned axial passage way. As the cap is threaded onto the housing body, the insert is compressed causing the diameter of the passageway through the insert to decrease. Spacing member 54 preferably takes the form of a spring and is positioned between the first coupler member 52 and the second coupler member 56. The ends of spacing member 54 may be releasably secured (or fixedly attached) to each of the coupler members.

FIG. 2 illustrates in more detail the construction of the implant deployment system 10 with the implant, coil 40, being positioned within catheter lumen 26 at catheter distal end 22. Delivery system 30 includes a tubular delivery member 60 having a distal end 62, a proximal end 64 and a lumen 66 extending therethrough. Delivery system 30 also includes a coupling assembly 70 having a tubular tip portion 72 with a distal end 73 and lumen 74, and an elongate actuator member 76 with proximal and distal ends 77 and 78 respectively. Tip portion 72 is shown positioned at the distal end 62 of delivery member 60 and secured to delivery member 60 preferably by laser welding but may take the form of any suitable joining technique such as soldering, spot welding, adhesives and ultrasonic welding. Actuator member 76 preferably takes the form of an elongate resilient nitinol wire although other materials and forms such as tubes or cables may be suitable. Elongate actuator member 76 is positioned within the lumen of delivery member 60 and has a tubular retaining member 80 fixedly attached to actuator member distal end 78 at securing joint 79. Securing joint 79 preferably takes the form of solder, but may take the form of any suitable joining technique such as UV curable adhesives, laser welds and ultrasonic welds to bond actuator member distal end 78 to retaining member 80. Retaining member 80 has a proximal and distal ends 82 and 84 respectively and includes a retaining portion 86 positioned at distal end 84. Retaining portion 86 may be joined to distal end 84 or integrally formed as a portion of the tubular wall of retaining member 80. Retaining portion 86 also includes a recessed region within the wall preferably taking the form of an aperture 88 that extends through the wall. Retaining member 80 is generally dimensioned to be slidably positioned within lumen 74 of tip portion 72 at distal end 73.

As previously discussed, the proximal end 44 of embolic coil 40 is releasably coupled to the distal end 32 of delivery system 30. More particularly, the proximal end 44 of embolic coil 40 is positioned within lumen 74 of tip portion 72 adjacent retaining member 80 of coupling assembly 70. Shown in FIG. 2, delivery system 30 is in a retracted configuration, engaging embolic coil 40, where retaining portion 86 of retaining member 80 is positioned within the lumen of tip portion 72 and coil proximal bead 45 is partially positioned within aperture 88. Tip portion 72 and retaining portion 86 cooperatively engage bead 45 of embolic coil 40 in an interlocking arrangement to thereby maintain the attachment of coupling assembly 70 to embolic coil 40. The actuator assembly 50 coupled to proximal end 34 of delivery system 30, operatively maintains the delivery system in a retracted configuration. In more detail, the first coupler member 52 is secured to proximal end 64 of delivery member 60 while the second coupler member 56 is secured to proximal end 77 of actuator member 76 and spacing member 54 is positioned between coupler members 52 & 56. The longitudinal spatial arrangement between the secured first and second coupler members 52 and 56 is such that when the coupling assembly 70 of delivery system 30 is in a retracted configuration, spacing member 54 is preferably placed in a slight state of compression. The spring force of spacing member 54 provides a bias to the maintain the coupling assembly 70 in a retracted configuration by simultaneously applying a proximally directed force to the proximal end 77 of actuator member 76 and a distally directed force to the proximal end 64 of delivery member 60.

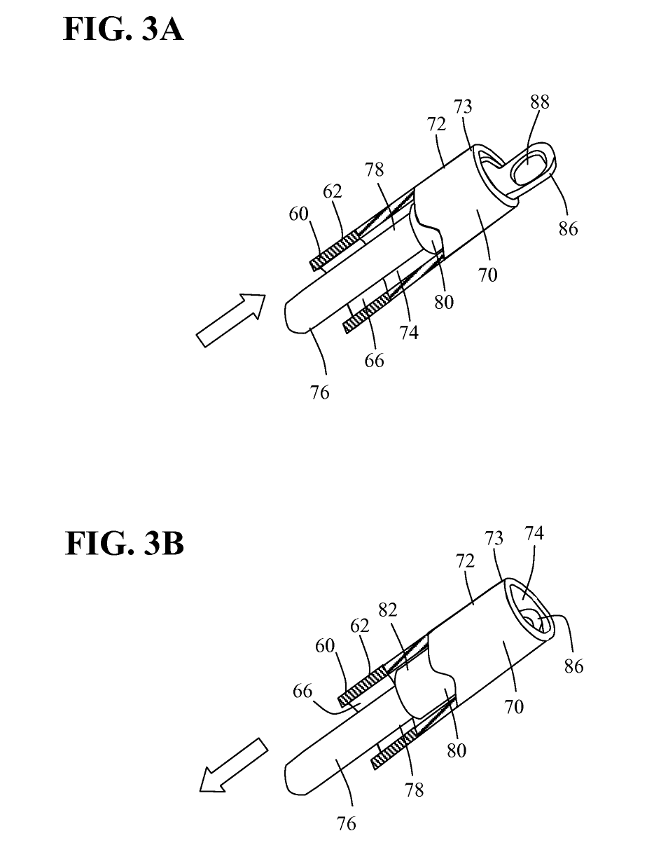

FIGS. 3A and 3B illustrate more detailed partial perspective views of coupling assembly 70 in extended and retracted configurations. FIG. 3A shows coupling assembly 70 placed in an extended configuration. Actuator assembly 50 is utilized to operatively move the coupling assembly between retracted and extended configurations. From the retracted configuration, advancing the secured second coupler member 56 towards the secured first coupler member 52, advances proximal end 77 of actuator member 76 distally relative to delivery member proximal end 64, thereby causing retaining member 80 to move distally relative to tip portion distal end 73. In the extended configuration, the retaining portion 86 of retaining member 80 extends distal to the tip portion distal end 73 and is suitably positioned to receive or release the proximal end of the embolic coil. From the extended configuration, secured second coupler member 56 is moved proximally relative to secured first coupler member 52, in turn, moving proximal end 77 of actuator member 76 proximally, thereby causing retaining portion 86 to move proximally towards tip portion distal end 73 as shown in FIG. 3B. In the retracted configuration, the retaining portion 86 of retaining member 80 is positioned within lumen 74 of tip potion 72.

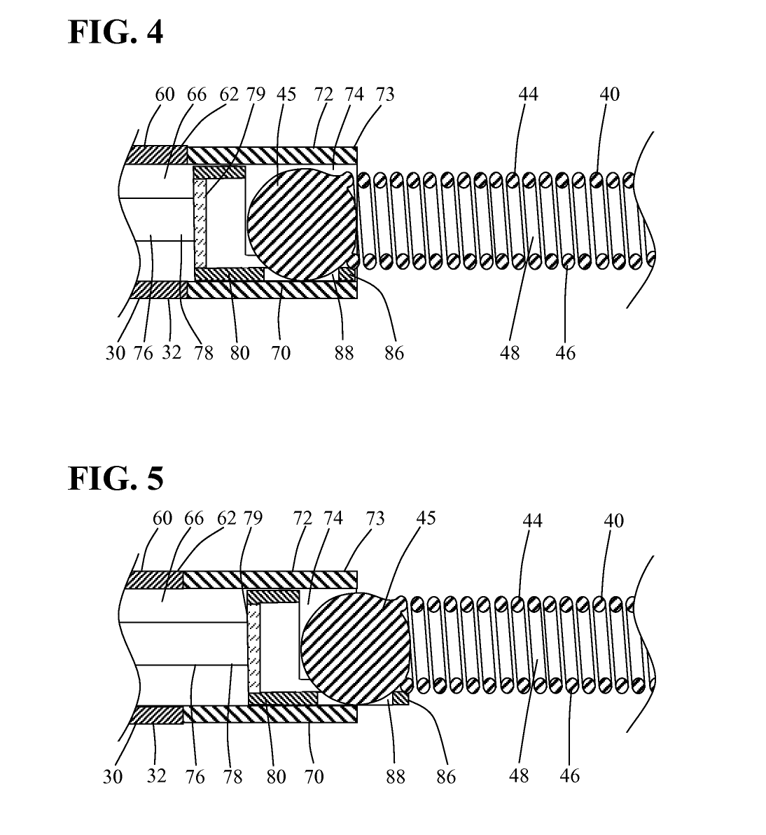

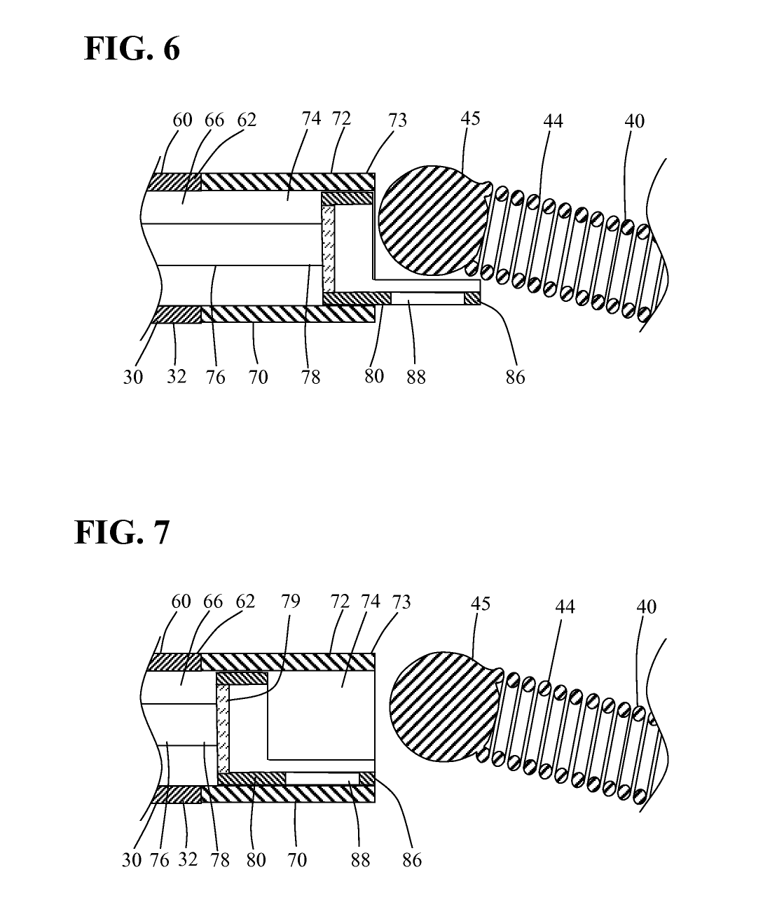

FIGS. 4 through 7 illustrate various relative positions of delivery system 30 and proximal end 44 of embolic coil 40 for clarification and discussion regarding loading coil 40 onto delivery system 30 or releasing coil 40 from delivery system 30. FIG. 4 depicts delivery system 30 in a retracted configuration engaged with embolic coil 40. Coil bead 45 of embolic coil 40 is engaged with delivery system 30 in an interlocking arrangement created by the cooperative efforts of tip portion 72 and retaining portion 86 of retaining member 80. As shown in FIG. 4, coil bead 45 is partially received by aperture 88 of retaining portion 86 and the diameter of coil bead 45 is slightly smaller than the diameter of tip portion lumen 74. The wall thickness of retaining portion 86 at the distal end of retaining member 80 in cooperation with the lumen 74 of tip portion 72 prevent coil bead 45 from exiting lumen 74 while in the retracted configuration. FIG. 5 illustrates the transition from the retracted configuration of coupling assembly 70 to the extended configuration. Advancing the secured second coupler member 56 towards the secured first coupler member 52, advances proximal end 77 of actuator member 76 distally relative to delivery member proximal end 64, thereby causing retaining member 80 to move distally relative to tip portion 72 such that the retaining portion 86 and coil bead 45 partially extend distal to distal end 73. By operatively placing coupling assembly 70 in the extended configuration (FIG. 6) retaining member 80 is positioned distal to its previous position in the retracted configuration and retaining portion 86 extends distal to distal end 73. In this configuration the tip portion 72 and retaining portion 86 no longer cooperate to create an interlocking arrangement with coil bead 45, thereby releasing coil 40 from delivery system 30. The distance retaining member 80 is moved from the retracted configuration to the extended configuration is dependent upon a number of factors including the dimensions of retaining portion 86, tip portion 32 and coil bead 45. This distance typically ranges from about 0.01 mm to about 5 mm with a preferred range of about 0.1 mm to 1.0 mm. FIG. 7 illustrates the distal end 32 of delivery system 30 positioned adjacent the released coil bead 45 at proximal end 44 of coil 40. More particularly, coupling assembly 70 is operatively placed in a retracted configuration so that coil bead 45 cannot be inadvertently re-engaged by retaining portion 86.

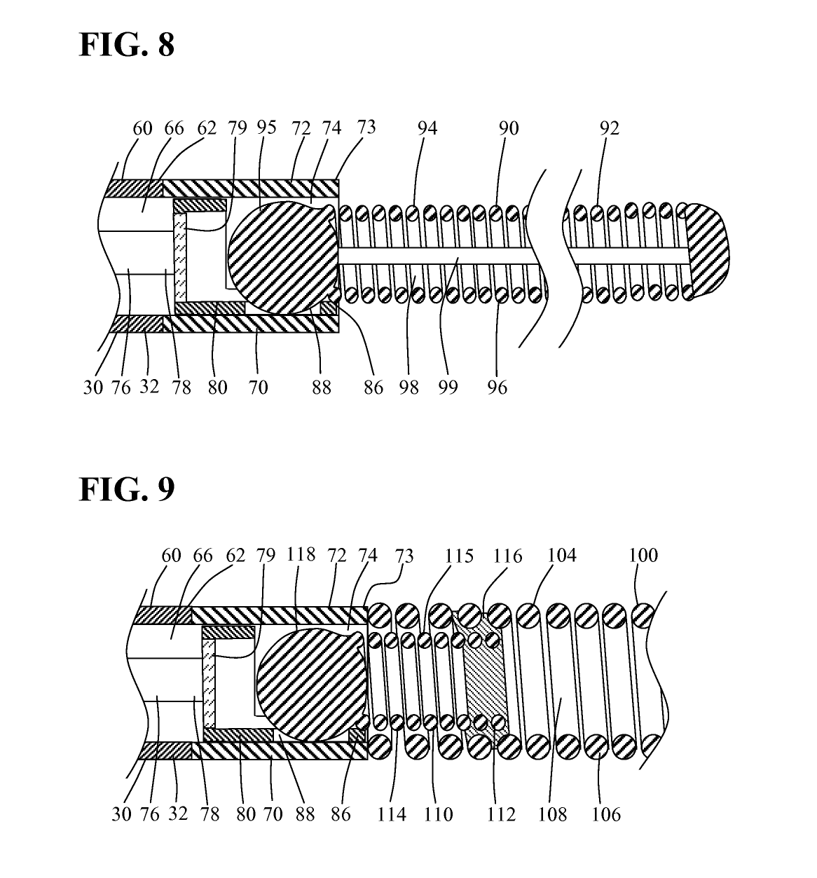

FIGS. 8 and 9 generally illustrate delivery system 30 engaged with alternate embodiments of embolic coils. FIG. 8 shows delivery system 30 releasably coupled to an embolic coil 90 having distal and proximal ends 92, 94 respectively and an engagement member 95 positioned at proximal end 94. Engagement member 95 preferably takes the form of a bead; however other shapes and geometries compatible with retaining portion 86 and tip portion 72 may also be suitable. Embolic coil 90 is formed by helically winding wire 96 thereby forming lumen 98. As previously discussed, embolic coils may have various shapes, sizes and modifications but are generally biocompatible and preferably radio-opaque. Embolic coil 90 includes a stretch resistant member 99 extending through lumen 98 that limits undesirable elongation of coil 90 during device manipulation. Stretch resistant member 99 is filamentous (preferably a wire having a small diameter ranging from about 0.0001″ to about 0.003″) and has one end coupled to distal end 92 and the other end fixedly attached to engagement member 95. Although engagement member 95 is positioned at proximal end 94 and fixedly coupled to coil 90 through stretch resistant member 99, engagement member 95 may optionally be fixedly coupled directly to proximal end 94 as shown. Engagement member 95 is releasably coupled to delivery system distal end 32. More particularly, engagement member 95 is secured by coupling assembly 70 in the retracted configuration whereby tip portion 72 and retaining portion 86 cooperatively form an interlocking arrangement with engagement member 95 within lumen 74 at distal end 73.

FIG. 9 shows delivery system 30 releasably coupled to an embolic coil 100 having distal end 102 (not shown) and proximal end 104. Embolic coil 100 is formed by helically winding wire 106 thereby forming lumen 108. As previously discussed, embolic coils may have various shapes, sizes and modifications but are generally biocompatible and preferably radio-opaque. Embolic coil 100 includes a headpiece member 110 having a distal end 112 and a proximal end 114. Headpiece member 110 takes the form of a flexible coil formed from wire 115. Headpiece member 110 is positioned within lumen 108 of coil 100 and the distal end 112 is fixedly coupled to coil 100 at joint member 116. Joint member 116 preferably takes the form of a solder however other materials and joining techniques such as adhesives and welding may be suitable secure headpiece distal end 112 to coil 100. Positioned at the proximal end 114 of headpiece 110 is engagement member 118. Engagement member 118 preferably takes the form of a bead; however other shapes and geometries compatible with retaining portion 86 and tip portion 72 may also be suitable. Engagement member 118 is positioned at proximal end 114 of headpiece 110 which is positioned at proximal end 104 of embolic coil 100. Engagement member 118 is releasably coupled to delivery system distal end 32. More particularly, engagement member 118 is secured by coupling assembly 70 in the retracted configuration whereby tip portion 72 and retaining portion 86 cooperatively form an interlocking arrangement with engagement member 118 within lumen 74 at distal end 73.

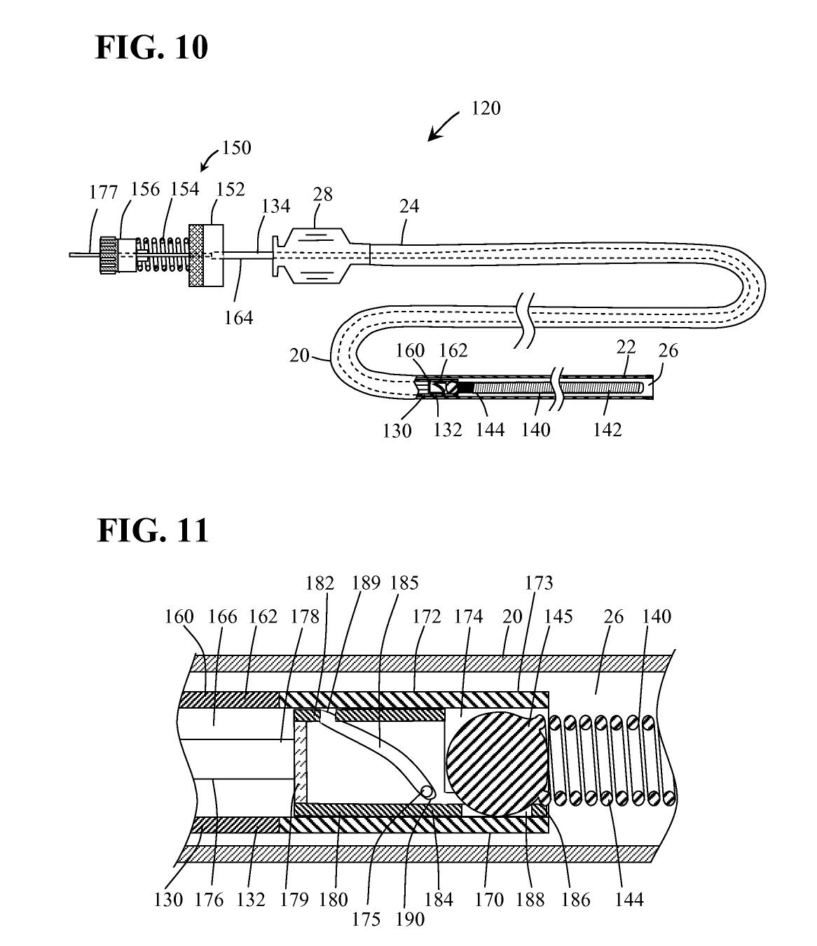

FIG. 10 generally illustrates an alternate embodiment of a medical implant deployment system 120 which includes delivery catheter 20 having a distal end 22, a proximal end 24, a lumen 26 extending therethrough and a catheter hub 28 affixed to proximal end 24, a delivery system 130 having a distal end 132 and a proximal end 134 and an embolic coil 140 having a distal end 142 and a proximal end 144 that is releasably coupled to the distal end 132 of delivery system 130. The proximal end 144 of the coil typically includes an enlarged bead 145 to facilitate coupling to delivery system 130, while the distal end 142 of the coil is typically rounded or beaded to make the coil end more atraumatic to the tissue encountered within the body. Delivery system 130 has a retracted configuration in which the coil 140 is securely coupled to the delivery system distal end 132 and an extended configuration in which the coupled coil 140 is released from distal end 132.

Embolic coil 140 is a medical implant of a general type suitable for use in occluding a vessel, duct or aneurysm and is generally formed from a primary coil of a helically wound wire 146, made from a material which is biocompatible and preferably radio-opaque. Suitable biocompatible materials include metals such as platinum, platinum alloys, stainless steel, nitinol, tantalum and gold and plastics such as nylons, polyesters, polyolefins and fluoropolymers. The wire usually has a circular cross-section, however, non-circular cross-sections, such as “D” shapes, may be used to form coils. The diameter of the wire may range from about 0.0001″ to about 0.010″ and is largely dependent upon the particular clinical application for the coil. The diameter of the primary coil is generally dependent upon the wire diameter and the diameter of the mandrel used for winding. The primary coil diameter typically ranges from about 0.002″ to about 0.060″ and is also dependent upon on the clinical application. The wound primary coil is typically removed from the mandrel leaving the coil with a lumen 148. In addition to the aforementioned method of winding a coil, there are other “mandrel-less” forming processes that are suitable for making primary coils that plastically deform the wire into coil. The formed primary coils may be further processed to have a secondary shape such as a helix, sphere, “flower”, spiral or other complex curved structure suited for implantation in a particular anatomical location. The secondary shape is imparted to the coil through thermal or mechanical means. Thermal means include forming the primary coil into a desired shape using a die or forming tool and then heat treating the coil to retain the secondary shape. Mechanical means include plastically deforming the primary coil into the desired shape or the use of a shaped resilient core wire inserted into the lumen of the primary coil to impart a shape to the coil. The length of the elongate primary coil typically ranges from about 0.1 cm to about 150 cm with a preferred range of about 0.5 cm to about 70 cm. Other variations of embolic coils suitable for use include stretch resistant coils, coils that incorporate a stretch resistant member(s) (within the coil lumen or exterior to the coil) that limits undesirable elongation of the primary coil during device manipulation and coated or modified coils that enhance occlusion through coil surface modifications, addition of therapeutics or volume filling materials (foams, hydrogels, etc.).

As depicted in FIG. 10, deployment system 120 may further include an actuator assembly 150 that is positioned proximal to proximal end 24 of catheter 20. Actuator assembly 150 includes a first coupler member 152, a spacing member 154 and a second coupler member 156. The first and second coupler members 152 and 156 typically take the form of commercially available rotating hemostatic valve (RHV) like assemblies. A typical RHV-like assembly includes a housing body, a threaded cap and a compressible insert. The housing body and threaded cap are typically formed of a rigid plastic such as polystyrene, ABS, nylon or polycarbonate while the insert is formed of an elastomeric material such as silicone or rubber. The assembled housing body, cap and insert all have a contiguous aligned axial passage way. As the cap is threaded onto the housing body, the insert is compressed causing the diameter of the passageway through the insert to decrease. Spacing member 154 preferably takes the form of a spring and is positioned between the first coupler member 152 and the second coupler member 156. The ends of spacing member 154 may be releasably secured (or fixedly attached) to each of the coupler members.

FIG. 11 illustrates in more detail the construction of the implant deployment system 120 with the implant, coil 140, being positioned within catheter lumen 26 at catheter distal end 22. Delivery system 130 includes a tubular delivery member 160 having a distal end 162, a proximal end 164 and a lumen 166 extending therethrough. Delivery system 130 also includes a coupling assembly 170 having a tubular tip portion 172, fixedly attached to distal end 162 of delivery member 160, with a distal end 173, a lumen 174 and a guide pin 175, an elongate actuator member 176 with proximal and distal ends 177 and 178 respectively, where distal end 178 is fixedly coupled via joint member 179 to tubular retaining member 180. Actuator member 176 preferably takes the form of an elongate resilient nitinol wire although other materials and forms such as tubes or cables may be suitable. Retaining member 180 has proximal and distal ends 182 and 184, a guide path 185 having a fixed longitudinal length that takes the form of a slot through the wall of retaining member 180 and includes a retaining portion 186 positioned at distal end 184. Retaining portion 186 may be joined to distal end 184 or integrally formed as a portion of the tubular wall of retaining member 180. Retaining portion 186 also includes a recessed region within the wall preferably taking the form of an aperture 188 that extends through the wall. Retaining member 180 is generally dimensioned to be slidably positioned within lumen 174 of tip portion 172 at distal end 173. Guide path 185 typically has a proximal end 189 and a distal end 190 which are offset longitudinally along the wall of retaining member 180 and define a guide path longitudinal length. Guide pin 175 of tubular tip portion 172 is generally received by guide path 185 of retaining member 180 such that the longitudinal distance retaining member 180 is allowed to travel is limited by the longitudinal length of guide path 185.

As previously discussed, the proximal end 144 of embolic coil 140 is releasably coupled to the distal end 132 of delivery system 130. More particularly, the proximal end 144 of embolic coil 140 is positioned within lumen 174 of tip portion 172 adjacent retaining member 180 of coupling assembly 170. Shown in FIG. 11, delivery system 130 is in a retracted configuration, engaging embolic coil 140, where retaining portion 186 of retaining member 180 is positioned within the lumen of tip portion 172 and coil proximal bead 145 is partially positioned within aperture 188. Tip portion 172 and retaining portion 186 cooperatively engage bead 145 of embolic coil 140 in an interlocking arrangement to thereby maintain the attachment of coupling assembly 170 to embolic coil 140. In the retracted configuration, guide pin 175 is generally positioned adjacent distal end 190 of guide path 185. The actuator assembly 150 coupled to proximal end 134 of delivery system 130, operatively maintains the delivery system in a retracted configuration. In more detail, the first coupler member 152 is secured to proximal end 164 of delivery member 160 while the second coupler member 156 is secured to proximal end 177 of actuator member 176 and spacing member 154 is positioned between coupler members 152 & 156. The longitudinal spatial arrangement between the secured first and second coupler members 152 and 156 is such that when the coupling assembly 170 of delivery system 130 is in a retracted configuration, spacing member 154 is preferably placed in a slight state of compression. The spring force of spacing member 154 provides a bias to the maintain the coupling assembly 170 in a retracted configuration by simultaneously applying a proximally directed force to the proximal end 177 of actuator member 176 and a distally directed force to the proximal end 164 of delivery member 160.

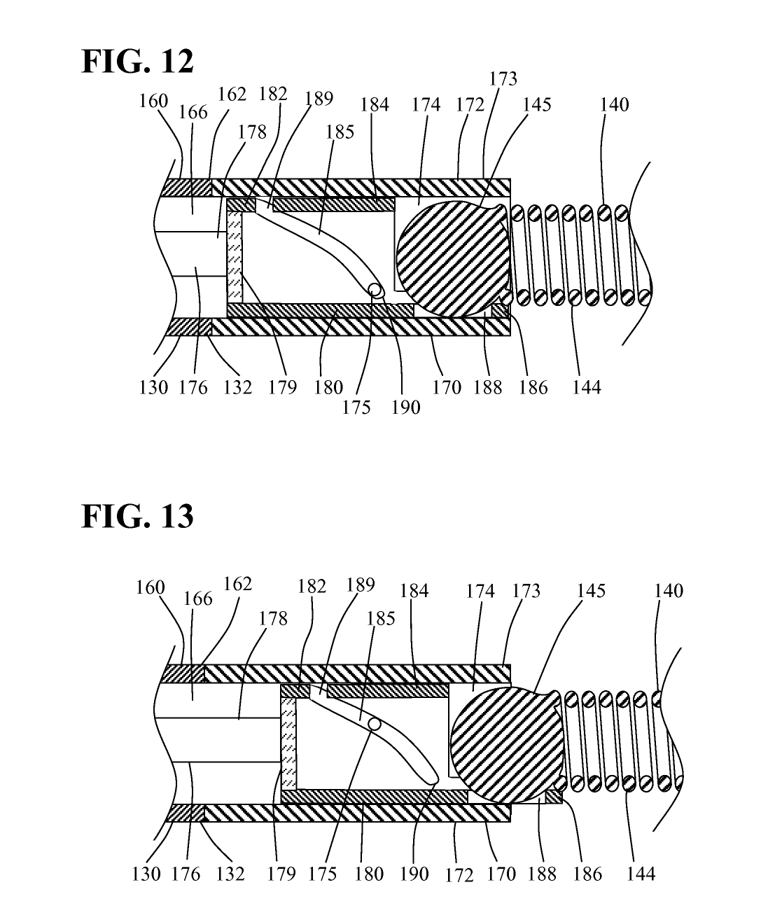

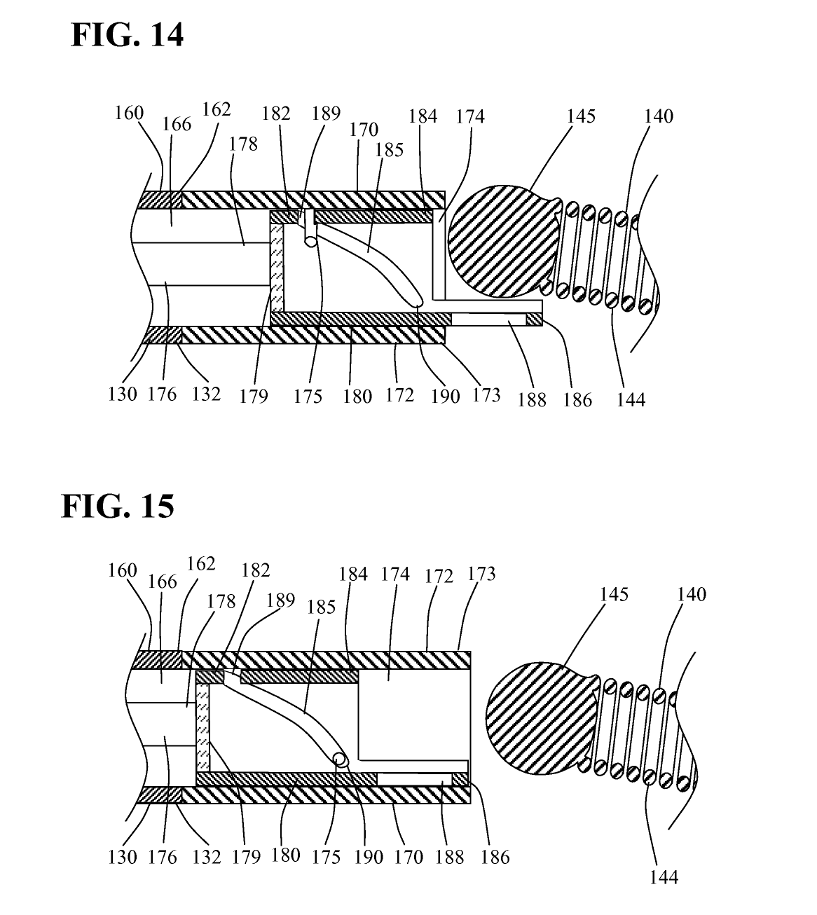

FIGS. 12 through 15 illustrate various relative positions of delivery system 130 and proximal end 144 of embolic coil 140 for clarification and discussion regarding the operation of delivery system 130 in loading coil 140 onto delivery system 130 or releasing coil 140 from delivery system 130. FIG. 12 depicts delivery system 130 in a retracted configuration engaged with embolic coil 140. Coil bead 145 of embolic coil 140 is engaged with delivery system 130 in an interlocking arrangement created by the cooperative efforts of tip portion 172 and retaining portion 186 of retaining member 180. As shown in FIG. 12, coil bead 145 is partially received by aperture 188 of retaining portion 186 and the diameter of coil bead 145 is slightly smaller than the diameter of tip portion lumen 174. The wall thickness of retaining portion 186 at the distal end of retaining member 80 in cooperation with the lumen 174 of tip portion 172 prevent coil bead 145 from exiting lumen 174 while in the retracted configuration. In the retracted configuration guide pin 175 of tubular tip portion 172 is generally received by guide path 185 of retaining member 180 and positioned adjacent the distal end 184 of retaining member 180. FIG. 13 illustrates the coupling assembly 170 of delivery system 130 transitioning from the retracted configuration to the extended configuration. Advancing the secured second coupler member 156 towards the secured first coupler member 152, advances proximal end 177 of actuator member 176 distally relative to delivery member proximal end 164, thereby causing retaining member 180 to move distally relative to tip portion 172 such that the retaining portion 186 and coil bead 145 partially extend distal to distal end 173. As coupling assembly 170 transitions from the retracted configuration to the extended configuration, guide path 185 of retaining member 180 moves distally relative to guide pin 175. Distal end 190 moves distally relative to guide pin 175, thereby positioning guide pin 175 longitudinally closer to proximal end 189 in relation to the position of guide pin 175 in the retracted configuration. By operatively placing coupling assembly 170 in the extended configuration (FIG. 14) retaining member 180 is positioned distal to its previous position in the retracted configuration and retaining portion 186 extends distal to distal end 173. Accordingly, in the extended configuration, guide pin 175 of tubular tip portion 172 is positioned adjacent proximal end 189 of guide path 185. In this configuration the tip portion 172 and retaining portion 186 no longer cooperate to create an interlocking arrangement with coil bead 145, thereby releasing coil 140 from delivery system 130. The distance retaining member 180 is longitudinally moved from the retracted configuration to the extended configuration is dependent upon a number of factors including the dimensions of retaining portion 186, tip portion 172 and coil bead 145 and is specifically controlled by the length of guide path 185 and the position of guide pin 175. This distance typically ranges from about 0.01 mm to about 5 mm with a preferred range of about 0.1 mm to 1.0 mm. FIG. 15 illustrates the distal end 132 of delivery system 130 positioned adjacent the released coil bead 145 at proximal end 144 of coil 140. More particularly, coupling assembly 170 is operatively placed in a retracted configuration so that coil bead 145 cannot be inadvertently re-engaged by retaining portion 186.

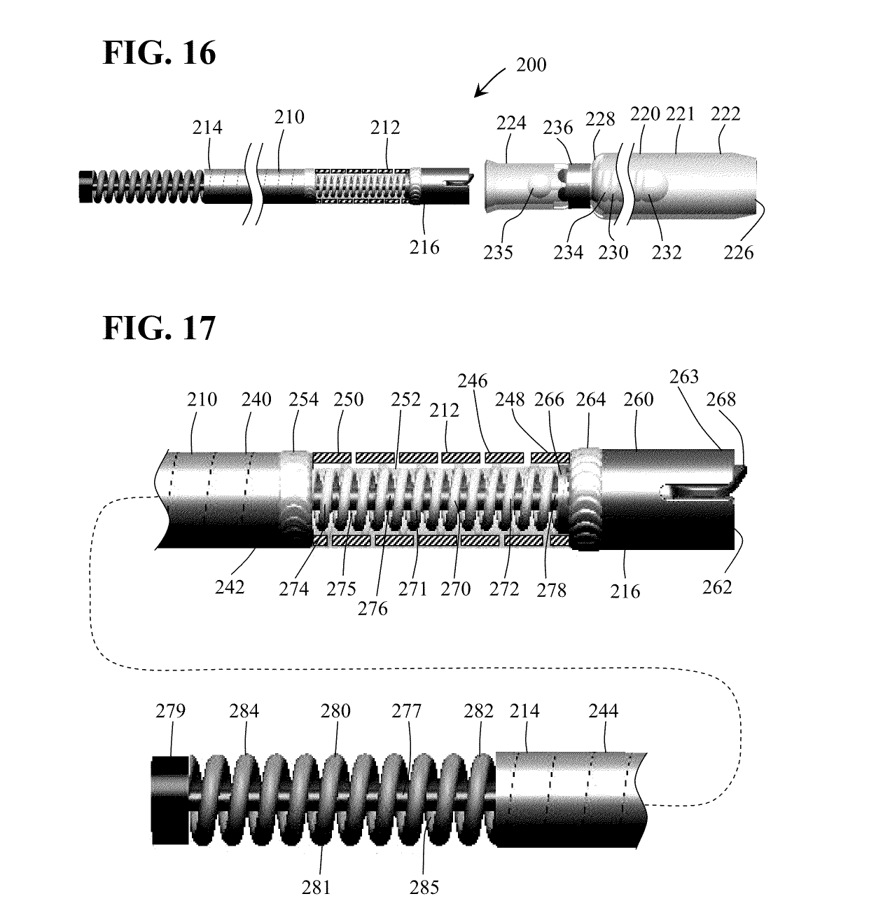

FIG. 16 generally illustrates another embodiment of a medical implant deployment system 200 which includes a delivery system 210 having a distal end 212, a proximal end 214 and a coupling assembly 216 positioned at distal end 212 and a coil introducer assembly 220 that includes a tubular coil introducer 221 having a distal end 222, a proximal end 224, a lumen 226 extending therethrough and a securing region 228 positioned between proximal end 224 and distal end 222, an embolic coil 230 having a distal end 232, a proximal end 234 and an engagement member 235 positioned at the proximal end 234 of embolic coil 230 and a securing member 236 that is positioned at securing region 228 of coil introducer 221. Embolic coil 230 is generally slidably positioned within the lumen 226 of introducer 221 wherein the coil distal end 232 is positioned distal to securing region 228 and the coil proximal end 234 is positioned within securing region 228. Securing member 236 is positioned at securing region 228 coincident with coil proximal end 234 and has an engagement configuration in which the luminal wall of introducer 221 in securing region 228 substantially restricts axial movement of coil proximal end 234 within introducer 221 and a release configuration in which the luminal wall of introducer 221 in securing region 228 allows axial movement of coil proximal end with reduced or minimal resistance.

The construction of delivery system 210 is illustrated in more detail in FIG. 17. Delivery system 210 includes a tubular delivery member 240 having a distal end 242, a proximal end 244 and a lumen extending therethrough. Delivery member 240 includes a marker coil 246 having a distal end 248, a proximal end 250 and a lumen 252 extending therethrough. Marker coil 246 is preferably formed from a radiopaque material to provide visibility of the delivery member distal end 242 under fluoroscopy. To provide for the largest possible diameter of lumen 252, marker coil 246 is preferably formed from flat wire or ribbon wire having a thin rectangular cross section. The marker coil proximal end 250 is fixedly coupled to delivery member 240 at distal end 242 by joint member 254. Coupling assembly 216 includes a tubular tip portion 260 having a through lumen 262 and distal end 263 is fixedly coupled to delivery member 240 at the marker coil distal end 248 by joint member 264. Joint members 254 and 264 preferably take the form of welds, although other forms such as adhesives, thermal bonds and other joining techniques may be suitable for joining components of delivery member 240. Coupling assembly 216 additionally includes tubular retaining member 266 having a retaining portion 268 positioned at the distal end of retaining member 266. Retaining member 266 is slidably positioned within lumen 262 of tubular tip portion 260.

Delivery system 210 further includes a distal actuator element 270 positioned within lumen 252 at the distal end 242 of delivery member 240. Distal actuator element 270 preferably takes the form of a coil spring formed from wire 271 and has a distal end 272, a proximal end 274 and a lumen 275 extending therethrough. Distal actuator element 270 distal end 272 is fixedly coupled to the proximal end of retaining member 266 while proximal end 274 is restricted from moving proximally. To restrict proximal movement of proximal end 274, the lumen of delivery member 240 at joint member 254 may have a diameter smaller than the outer diameter of actuator element 270 or proximal end 274 may optionally be secured by joint member 254. An elongate actuator member 276 having proximal and distal ends 277 and 278 respectively, where distal end 278 is fixedly coupled to tubular retaining member 266, is slidably positioned within the lumen of delivery member 240 at distal end 242. More particularly, actuator member 276 is coaxially positioned within lumen 275 of distal actuator element 270. Actuator member 276 preferably takes the form of an elongate resilient nitinol wire although other materials and forms such as tubes or cables may be suitable. Proximal end 277 of actuator member 276 extends proximal to delivery member proximal end 244 and includes a fixedly attached stop member 279.

Delivery system 210 also includes a proximal actuator element 280 positioned proximal to delivery member proximal end 244. Proximal actuator element 280 preferably takes the form of a coil spring formed from wire 281 and has a distal end 282, a proximal end 284 and a lumen 285 extending therethrough. More particularly, proximal actuator element 280 is coaxially positioned about proximal end 277 of actuator member 276. The distal end 282 of proximal actuator element 280 is fixedly coupled to the proximal end 244 of delivery member 240 while proximal end 284 is fixedly coupled to stop member 279.

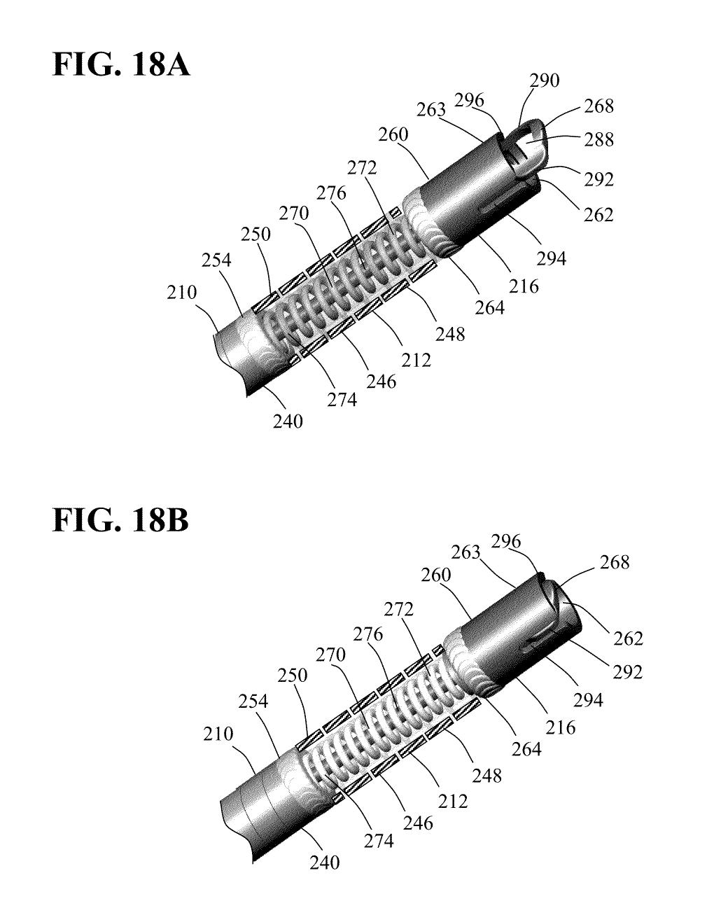

Delivery system 210 has two general configurations, which include a retracted configuration and an extended configuration, and can be operated to transition between the two configurations. FIGS. 18A and 18B generally illustrate the distal end 212 of delivery system 210 in the extended and retracted configurations. In the extended configuration retaining member 266 of coupling assembly 216 is positioned such that retaining portion 268 extends distal to tip portion distal end 263 as shown in FIG. 18A. Retaining portion 268 may further include a recessed region within the wall preferably taking the form of an aperture 288 that extends through the wall and guide members 290 and 282 adjacent aperture 288. Guide members 290 and 292 typically take the form of extension members that are adapted to be received by guide paths 294 and 296 of tubular tip portion 260 which take the form of slots through the wall. Generally, distal actuator element 270 is normally biased to exert a force on retaining member 266 to place coupling assembly 216 of delivery system 210 in the extended configuration. The extended configuration positions retaining portion 268 in a state to receive or release the proximal end of an embolic coil. FIG. 18B shows the distal end 212 of delivery system 210 in the retracted configuration. In the retracted configuration retaining member 266 of coupling assembly 216 is generally positioned such that retaining portion 268 is positioned within lumen 262 of tip portion 260 and does not substantially extend distal to tip portion distal end 263. Generally, proximal actuator element 280 is normally biased to exert a force on actuator member 276 coupled to retaining member 266 to place coupling assembly 216 of delivery system 210 in the retracted configuration. Delivery system 210 is preferably normally biased in the retracted configuration and maintained in this configuration because the force applied to actuator member 276 by proximal actuator element 280 is typically larger than the force applied to retaining member 266 by distal actuator element 270.

To operatively transition delivery system 210 between the retracted and extended configurations, actuator member proximal end 277 is moved proximally or distally relative to delivery member 240 and is dependent upon the starting configuration. For instance, from the extended configuration, delivery system 210 may be transitioned to the retracted configuration by retracting actuator member proximal end 277 relative to delivery member 240 thereby causing actuator member distal end 178 to retract retaining member 266 proximally, retracting retaining portion 268 within lumen 262 of tip portion 260. From the retracted configuration, delivery system 210 may be transitioned to the extended configuration by advancing actuator member proximal end 277 relative to delivery member 240 thereby causing actuator member distal end 178 to advance retaining member 266 distally, extending retaining portion 268 distal to tip portion distal end 263.

In general, elongate devices such as delivery system 210 that are operated by retracting or advancing a small diameter wire, such as actuator member 276, from the proximal end of the system, may potentially encounter movement difficulties when placed in a tortuous environment comprising multiple bends. The difficulties are typically encountered while attempting to advance the wire, rather than retracting the wire, due to force transmission losses encountered at bend regions. To address the potential difficulties, the distal actuator element 270 of delivery system 210 assists in the advancement of actuator member 276. Since actuator element 270 is normally biased to advance retaining member 266 distally, to extend retaining portion 268 distal to tip portion distal end 263, operating delivery system 210 to transition from the retracted configuration to the extended configuration, simultaneously applies a proximally located push force and a distally located pull force to actuator member 276 thereby minimizing the effect of transmission losses.

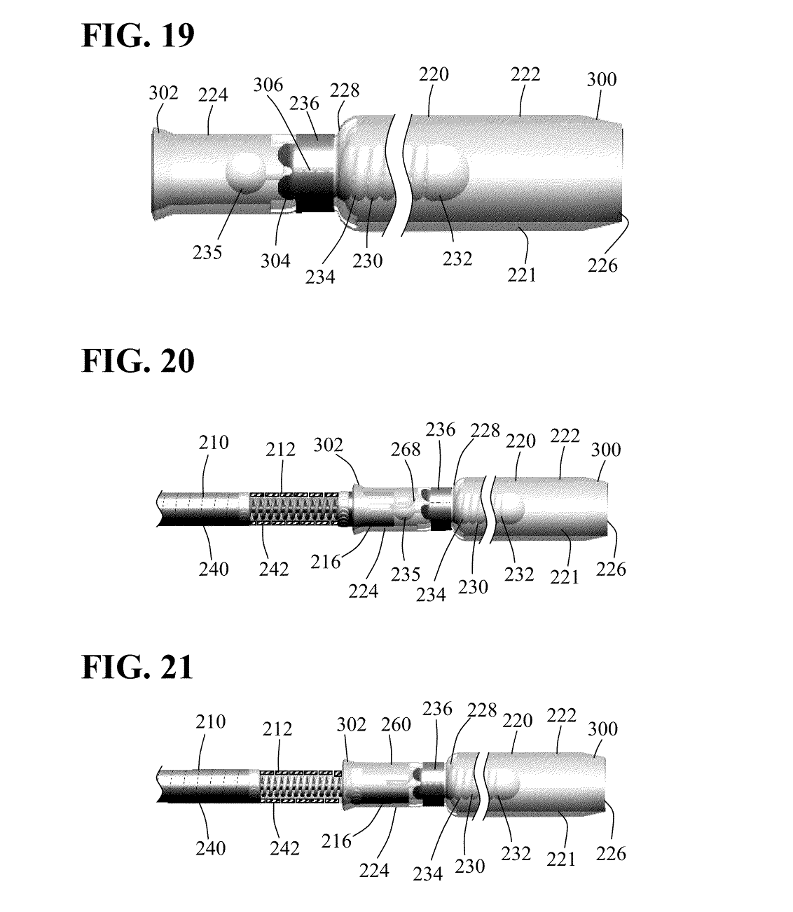

The aforementioned embodiments of delivery systems are suitable for use in engaging, delivering, deploying and releasing multiple medical implants, such as embolic coils, to a target site within the body. An implant assembly and method enabling the engagement, delivery, deployment and release of a medical implant at a target site by a delivery system is provided and described herein. FIG. 19 illustrates coil introducer assembly 220 of deployment system 200 in more detail. As previously discussed, coil introducer assembly 220 includes tubular coil introducer 221 having distal end 222, proximal end 224, lumen 226 extending therethrough and securing region 228 positioned between proximal end 224 and distal end 222, embolic coil 230 having distal end 232, proximal end 234 and engagement member 235 positioned at the proximal end 234 of embolic coil 230 and securing member 236 that is positioned at securing region 228 of coil introducer 221. Coil introducer 221 preferably takes the form of an elongate extruded polymeric transparent or translucent tube; however other materials and tubular construction techniques may be suitable. Introducer lumen 226 at proximal end 224 preferably has a lumen diameter which is smaller than the lumen diameter at distal end 222. The diameter transition of coil introducer lumen 226 from the smaller diameter at proximal end 224 to the larger diameter at distal end 222 typically occurs in securing region 228 of coil introducer 221.

Coil introducer assembly 220 performs several key functions which include retaining embolic coil 230 within lumen 226, enabling the introduction and engagement of delivery system 210 to coil 230 and enabling the transfer of coil 230 and delivery system 210 from introducer 221 to the lumen of a delivery catheter. To facilitate these functions, coil introducer 221 further includes a tapered tip 300 positioned at distal end 222 and a flared tip 302 positioned at proximal end 224. Tapered tip 300 and flared tip 302 may be formed using any number of commercially available known forming techniques including grinding and heat forming. As previously discussed, to retain embolic coil 230 within lumen 226 of introducer 221, proximal end 234 is positioned within securing region 228. Securing member 236 is positioned in coil introducer securing region 228 and has an engagement configuration to restrict coil movement and a release configuration to allow coil movement. The securing member may take many different forms including rotating hemostasis valves (RHV), elastic bands and removable clamping structures. The securing member may also be integrally formed with the introducer and include a plurality of slots through the wall of the introducer in the securing region and thermoforming the wall portion to provide the initial engagement configuration which then flex upon sufficient application of force from the delivery system to provide the release configuration. Preferably, securing member 236 takes the form of a heat shrink tubing to apply the compressive force in the engagement configuration. Securing member 236 also preferably includes tab member 304 and perforated region 306 to facilitate transitioning from the engagement configuration to the release configuration. In the engagement configuration, securing member 236 compresses introducer 221 in securing region 228 such that the wall of introducer 221 contacts proximal end 234, thereby applying sufficient force to restrict axial movement of coil 230 within introducer 221.

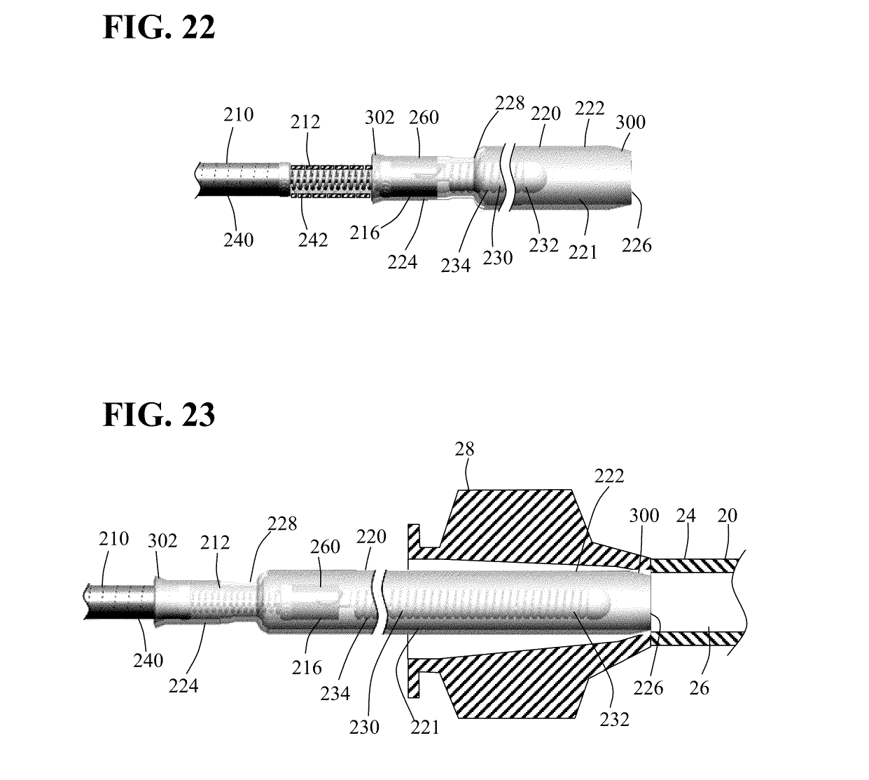

With securing member 236 in the engagement configuration to restrict movement of coil 230, delivery system 210 may be inserted into lumen 226 of introducer 221 at proximal end 224. Lumen 226 at flared tip 302 has a large tapering diameter to accommodate the introduction of distal end 212 into introducer 221. Coupling assembly 216 of delivery system 210 is positioned adjacent coil proximal end 234 within lumen 226. To couple delivery system 210 and embolic coil 230, coupling assembly 216 is placed in the extended configuration such retaining member 266 extends distal to tip portion 260. Retaining portion 268 is positioned in contact with engagement member 235 of coil proximal end 234 as illustrated in FIG. 20. Delivery system coupling assembly 216 is then operatively placed in the retracted configuration to secure engagement member 235 within tip portion 260 as shown in FIG. 21, thereby coupling delivery system 210 to embolic coil 230. Once the delivery system is securely coupled to the coil, securing member 236 may be transitioned to the release configuration. To place securing member 236 in the release configuration tab member 304 is pulled to tear the wall of the heat shrink tubing along perforated region 306 to thereby remove securing member 236 and the compressive force applied in the engagement configuration as shown in FIG. 22.

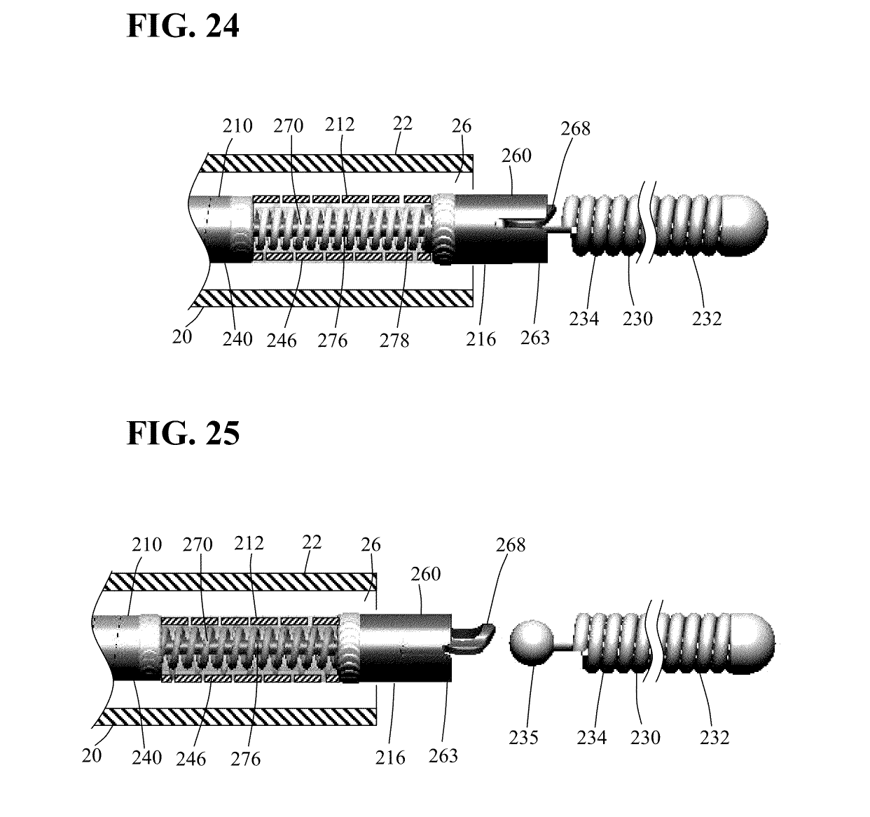

FIG. 23 illustrates the introduction of delivery system 210 engaged with embolic coil 230 into delivery catheter 20. Distal end 222 of coil introducer 221 is inserted within the delivery catheter hub 28, such that tapered tip 300 is positioned adjacent lumen 26 at catheter proximal end 24. In this position, delivery system 210 may be advanced relative to introducer 221 to thereby advance embolic coil distal end 232 towards tapered tip 300 as shown in FIG. 23. Continued distal advancement of delivery system 210 advances embolic coil 230 into lumen 26 of catheter 20. With delivery catheter distal end 22 positioned at a target site within the body, delivery system 210 is advanced through catheter 20 such that embolic coil 230 exits lumen 26 at catheter distal end 22, as illustrated in FIG. 24. After properly positioning embolic coil 230 at the target site, delivery system 210 may be operatively placed in the extended configuration to thereby release coil 230 at the target site. Delivery system 210 may then be removed from the catheter and reloaded with an additional coil according to the aforementioned procedures as needed.

Numerous modifications exist that would be apparent to those having ordinary skill in the art to which this invention relates and are intended to be within the scope of the claims which follow.

That which is claimed is:

1. An embolic coil delivery device for use in placing a coil at a preselected site within a vessel or lumen comprising:
an elongated flexible delivery member having proximal and distal ends and a lumen extending therethrough,
a coupling assembly positioned at the distal end of said delivery member including a tubular tip element having proximal and distal ends and a lumen extending therethrough, wherein said proximal end is fixedly coupled to the distal end of said delivery member, a tubular retaining member having proximal and distal ends and a retaining portion positioned at said retaining member distal end, wherein said retaining member is slidably positioned within the lumen of said tubular tip element,
said coupling assembly having a first configuration wherein said retaining portion of said retaining member extends distal to the distal end of said tip element and is adapted to receive or release the proximal end of an embolic coil and a second configuration wherein said retaining portion is positioned within the lumen of said tip element and said retaining portion and tip element are adapted to cooperatively retain the proximal end of an embolic coil, said coupling assembly being moveable between said first and second configurations, and
an elongate flexible resilient actuator member having proximal and distal ends and being slidably positioned within the lumen of said delivery member, said actuator member distal end being fixedly attached to said proximal end of said retaining member and said actuator member proximal end extends proximal to said delivery member proximal end, such that longitudinal movement of said actuator member proximal end operatively moves said coupling assembly between said first and second configurations.

2. An embolic coil delivery device according to claim 1 further comprising a first spring member having proximal and distal ends and a lumen extending therethrough, said first spring member being adjacent said actuator member and positioned at the distal end of said delivery member, said first spring member distal end being coupled to said retaining member proximal end and said first spring member proximal end is restricted from moving proximally.

3. An embolic coil delivery device according to claim 1 further comprising a first spring member having proximal and distal ends and a lumen extending therethrough, said first spring member is positioned adjacent the proximal end of said actuator member wherein the distal end of said first spring member is coupled to the proximal end of said delivery member and the proximal end of said first spring member is coupled to the proximal end of said actuator member.

4. An embolic coil delivery device according to claim 2 wherein said first spring member is normally biased to position said coupling assembly in said first configuration.

5. An embolic coil delivery device according to claim 3 wherein said first spring member is normally biased to position said coupling assembly in said second configuration.

6. An embolic coil delivery device according to claim 2 further comprising a second spring member having proximal and distal ends and a lumen extending therethrough, said second spring member is coaxially arranged about the proximal end of said actuator member wherein the distal end of said second spring member is fixedly coupled to the proximal end of said delivery member and the proximal end of said second spring member is fixedly coupled to the proximal end of said actuator member.

7. An embolic coil delivery device according to claim 6 wherein said first spring member is normally biased to position said coupling assembly in said first configuration and said second spring member is normally biased to place said coupling assembly in said second configuration.

8. An embolic coil delivery device according to claim 7 wherein said second spring member applies more force than said first spring member to said coupling assembly.

9. An embolic coil deployment system for use in placing a coil at a preselected site within a vessel or lumen comprising:
a flexible catheter having proximal and distal ends and a lumen extending therethrough,
an elongated flexible delivery member having proximal and distal ends and a lumen extending therethrough, said delivery member being positioned within the lumen of said catheter,
an embolic coil having proximal and distal ends, said coil being positioned within the lumen of said catheter and said coil being releasably coupled to said delivery member,
a coupling assembly positioned at the distal end of said delivery member and operatively engaged with the proximal end of said coil, including a tubular tip element having proximal and distal ends and a lumen extending therethrough, wherein said tip element proximal end is fixedly coupled to the distal end of said delivery member, a tubular retaining member having proximal and distal ends and a retaining portion positioned at said retaining member distal end, wherein said retaining member is slidably positioned within the lumen of said tubular tip element,
said coupling assembly having a first configuration wherein said retaining portion of said retaining member extends distal to the distal end of said tip element and is adapted to receive or release the proximal end of said embolic coil and a second configuration wherein said retaining portion is positioned within the lumen of said tip element and said retaining portion and tip element are adapted to cooperatively retain the proximal end of said embolic coil, said coupling assembly being moveable between said first and second configurations, and
an elongate flexible resilient actuator member having proximal and distal ends and being slidably positioned within the lumen of said delivery member, said actuator member distal end being fixedly attached to said proximal end of said retaining member and said actuator member proximal end extends proximal to said delivery member proximal end, such that longitudinal movement of said actuator member proximal end operatively moves said coupling assembly between said first and second configurations.

10. An embolic coil deployment system according to claim 9 further comprising a distal spring member having proximal and distal ends and a lumen extending therethrough, said distal spring member being adjacent said actuator member and positioned within the lumen of said delivery member distal end, said distal spring member distal end being coupled to said retaining member proximal end and said distal spring member proximal end is restricted from moving proximally.

11. An embolic coil deployment system according to claim 9 further comprising a proximal spring member having proximal and distal ends and a lumen extending therethrough, said proximal spring member is coaxially arranged about the proximal end of said actuator member wherein the distal end of said proximal spring member is coupled to the proximal end of said delivery member and the proximal end of said proximal spring member is coupled to the proximal end of said actuator member.

12. An embolic coil deployment system according to claim 9 wherein said retaining portion includes an aperture through the wall and said coil proximal end is partially received by said aperture.

13. An embolic coil deployment system according to claim 9 wherein said coil proximal end comprises a bead.

14. An embolic coil deployment system according to claim 9 wherein said retaining member includes at least one guide member, said tubular tip element includes at least one guide slot, and said guide member is received by said guide slot.

15. An embolic coil deployment system according to claim 9 wherein said tubular tip element includes at least one guide member, said retaining member includes at least one guide slot, and said guide member is received by said guide slot.

16. An embolic coil deployment system for use in placing a coil at a preselected site within a vessel or lumen comprising:
a flexible catheter having proximal and distal ends and a lumen extending therethrough,
an elongated flexible delivery member having proximal and distal ends and a lumen extending therethrough, said delivery member being positioned within the lumen of said catheter,
an embolic coil having proximal and distal ends and an engagement member positioned at said proximal end, said coil being positioned within the lumen of said catheter and said coil being releasably coupled to said delivery member,
a coupling assembly positioned at the distal end of said delivery member including a tubular tip element having proximal and distal ends and a lumen extending therethrough, wherein said tip element proximal end is fixedly coupled to the distal end of said delivery member, a tubular retaining member having proximal and distal ends and a retaining portion positioned at said retaining member distal end, wherein said retaining member is slidably positioned within the lumen of said tubular tip element,
said coupling assembly having a first configuration wherein said retaining portion of said retaining member extends distal to the distal end of said tip element and is adapted to receive or release the proximal end of said embolic coil and a second configuration wherein said retaining portion is positioned within the lumen of said tip element and said retaining portion and tip element are adapted to cooperatively retain the proximal end of said embolic coil, said coupling assembly being moveable between said first and second configurations,
said coupling assembly being operatively engaged with the proximal end of said coil whereby said engagement member is positioned adjacent said retaining portion within the lumen of said tip element,
an elongate flexible resilient actuator member having proximal and distal ends and being slidably positioned within the lumen of said delivery member, said actuator member distal end being fixedly attached to said proximal end of said retaining member and said actuator member proximal end extends proximal to said delivery member proximal end, such that longitudinal movement of said actuator member proximal end operatively moves said coupling assembly between said first and second configurations,
a distal spring member having proximal and distal ends and a lumen extending therethrough, said distal spring member being adjacent said actuator member and positioned within the lumen of said delivery member distal end, said distal spring member distal end being coupled to said retaining member proximal end and said distal spring member proximal end is restricted from moving proximally, and
a proximal spring member having proximal and distal ends and a lumen extending therethrough, said proximal spring member is coaxially arranged about the proximal end of said actuator member wherein the distal end of said proximal spring member is coupled to the proximal end of said delivery member and the proximal end of said proximal spring member is coupled to the proximal end of said actuator member.

17. An embolic coil deployment system according to claim 16 wherein said retaining portion includes an aperture through the wall and said engagement member is partially received by said aperture.

18. An embolic coil deployment system according to claim 16 wherein said engagement member comprises a bead.

19. An embolic coil deployment system according to claim 16 wherein said retaining portion includes at least one guide member, said tubular tip element includes at least one guide slot, and said guide member is received by said guide slot.

20. An embolic coil deployment system according to claim 16 wherein said tubular tip element includes at least one guide member, said retaining member includes at least one guide slot, and said guide member is received by said guide slot.
